(19) United States
(12) Patent Application Publication
(10) Pub. No.: US 2023/0363741 A1
(43) Pub. Date: Nov. 16, 2023

(54) ULTRASOUND DIAGNOSTIC SYSTEM

(71) Applicant: AIDOT INC., Seoul (KR)

(72) Inventor: Jae Hoon JEONG, Seongnam-si (KR)

(73) Assignee: AIDOT INC., Seoul (KR)

(21) Appl. No.: 18/026,001

(22) PCT Filed: Sep. 6, 2021
(86) PCT No.: PCT/KR2021/012018

§ 371 Date: Mar. 13, 2023

(30) Foreign Application Priority Data
Sep. 15, 2020 (KR) 10-2020-0118622

(51) Int. Cl. A61B 8/08; G06T 7/00; G06T 7/90

(52) U.S. Cl. CPC A61B 8/5223; G06T 7/0014; G06T 7/90; G06T 2207/20084; G06T 2207/10132

(57) ABSTRACT

The present invention relates to an ultrasound diagnostic system using an artificial neural network which is capable of providing convenience in operating a diagnostic device by performing guidance such that an ultrasound image of a carotid artery, a thyroid, a breast, a femoral vein, or a medium vein may be acquired at an optimal position. The ultrasound diagnostic system includes a diagnostic part search unit which finds a diagnostic part (carotid artery, thyroid, femoral vein, medium vein, or breast) from input images and is configured to represent and output at least the diagnostic part in a color differentiated from that of tissue, and an automatic diagnosis unit which diagnoses whether the diagnostic part is abnormal with respect to an image of the diagnostic part found by the diagnostic part search unit based on a first artificial neural network and is configured to output a diagnosis result.

### TECHNICAL FIELD

The present invention relates to an ultrasound diagnostic system and, more particularly, to a system for diagnosing an abnormal symptom of an ultrasound diagnostic part using one or more artificial neural networks.

### BACKGROUND ART

Ultrasound is a type of elastic wave. Accordingly, when the ultrasound propagates into the human body, the ultrasound is reflected, transmitted, or absorbed at an interface of a medium according to physical characteristics of the human tissue, and thus, an amplitude thereof is sometimes attenuated. When the characteristics of the ultrasound are used, an image of internal tissues of the human body can be acquired, and the size or characteristics of the tissue can be determined from the image so that ultrasound diagnostic devices are widely used in health care industries.

Carotid ultrasound, breast ultrasound, thyroid ultrasound, and deep vein thrombosis ultrasound (for a femoral vein or medium vein) are widely known for diagnosing whether a part of a body is abnormal using an ultrasound diagnostic device.

For reference, a carotid artery or carotid is an artery that passes through a neck and enters a face and a skull and is mainly divided into an external carotid artery and an internal carotid artery. The external carotid artery mainly supplies blood to the skin and muscles outside the skull, and the internal carotid artery supplies blood to the brain and nerve tissue inside the skull.

Even when the external carotid artery is narrowed or blocked, there is no particular problem because a relatively abundant amount of blood is supplied through other blood vessels. However, when the internal carotid artery is narrowed or blocked, the supply of blood to the brain may be reduced, and fatty tissues deposited (accumulated and attached) on a wall of the internal carotid artery may be separated to flow to a distal end of a brain blood vessel and to block the blood vessel. The narrowing of the carotid artery including the internal carotid artery is referred to as carotid artery stenosis, which reduces a blood flow or blocks blood vessels such as to cause an ischemic stroke. Therefore, patients with carotid artery stenosis are treated to prevent and treat a stroke.

One of the methods of diagnosing and examining carotid artery stenosis is a carotid ultrasound. The carotid ultrasound is a simple test for observing the presence or absence of plaque, blood flow, a blood vessel thickness, or the like in a carotid artery and has advantages in that it takes a short time for a test and test costs are low.

However, there is a disadvantage in that, only when an examiner has sufficient skill and knowledge can the examiner perform the test. In addition, there are disadvantages in that, even for the same ultrasound image, there may be difference in reading ability between examiners, and even an experienced examiner may have a low reading ability for a symptom appearing subtly in an ultrasound image, which increases the probability of misdiagnosis.

In particular, in order to accurately diagnose an abnormal symptom of a carotid artery, there is a need for a method of acquiring an ultrasound image of the carotid artery at an optimal position, but unless an examiner is an experienced professional, it is not easy for the examiner to move an ultrasound probe to a position at which an optimal ultrasound image may be acquired. This is a common problem in various ultrasound diagnoses using thyroid ultrasound and breast ultrasound as well as carotid ultrasound.

Accordingly, there is a need for a new type of ultrasound diagnostic system which can perform guidance to a position at which an ultrasound image, which is necessary for accurately diagnosing an abnormal symptom of a body part, may be acquired, thereby being conveniently used by anyone. In addition, there is a need for a new type of ultrasound diagnostic system which can accurately detect even a symptom appearing subtly in an ultrasound image to accurately predict and diagnose an abnormal symptom of a body part.

### RELATED ART DOCUMENTS

### Patent Documents

(Patent Document 1) Korean Patent Publication No. 10-2009840

### SUMMARY OF INVENTION

### Technical Problem

The present invention is directed to providing an ultrasound diagnostic system using an artificial neural network which is capable of automatically and accurately diagnosing abnormal symptoms in a carotid artery, a thyroid, a femoral vein, a medium vein, and a breast irrespective of an examiner.

The present invention is also directed to providing an ultrasound diagnostic system using an artificial neural network which is capable of providing convenience in operating a diagnostic device by performing guidance such that ultrasound images of a carotid artery, a thyroid, a femoral vein, a medium vein, and a breast may be acquired at an optimal position.

The present invention is also directed to providing an ultrasound diagnostic system using an artificial neural network which is capable of selecting only an ultrasound image of a carotid artery, a thyroid, a femoral vein, a medium vein, or a breast from input images to automatically diagnose whether the carotid artery, the thyroid, the femoral vein, the medium vein, or the breast is abnormal.

The present invention is also directed to providing an ultrasound diagnostic system using an artificial neural network which is capable of searching for blood vessel plaque that is likely to develop into a floating thrombus, thereby providing notification of the possibility of a stroke in advance.

The present invention is also directed to providing an ultrasound diagnostic system using an artificial neural network which is capable of automatically diagnosing whether at least one diagnostic part of a carotid artery, a thyroid, a femoral vein, a medium vein, and a breast is abnormal and capable of differentiating and displaying a risk of a diagnostic part (carotid artery, thyroid, femoral vein, medium vein, or breast) in multiple stages.

The present invention is also directed to providing an ultrasound diagnostic system using an artificial neural network which is capable of accurately and automatically diagnosing whether at least one of a carotid artery, a thyroid, a femoral vein, a medium vein, and a breast is abnormal using one or more artificial neural networks or which is capable of accurately and automatically diagnosing whether a carotid artery, a thyroid, a femoral vein, a medium vein, or a breast is abnormal with respect to an ultrasound image transmitted from a remote site to provide notification.

### Solution to Problem

According to an embodiment of the present invention, an ultrasound diagnostic system includes
a diagnostic part search unit which finds a diagnostic part from input images and is configured to represent and output at least the diagnostic part in a color differentiated from that of tissue, and
an automatic diagnosis unit which diagnoses whether the diagnostic part is abnormal with respect to an image of the diagnostic part found by the diagnostic part search unit based on a first artificial neural network and is configured to output a diagnosis result.

The diagnostic part search unit may include
a second artificial neural network pretrained to select only an ultrasound image of any one of a carotid artery, a thyroid, a breast, a femoral vein, and a medium vein from the input images and extract any one part of a carotid artery part, a thyroid part, a breast part, a femoral vein part, and a medium vein part from the selected ultrasound image.

The second artificial neural network may be an artificial neural network trained to select and display carotid ultrasound images in both a longitudinal direction and a lateral direction.

When the diagnosis result is abnormal,
the automatic diagnosis unit may diagnose a risk from an image of any one part of the carotid artery part, the thyroid part, the breast part, the femoral vein part, and the medium vein part using a pretrained third artificial neural network and may be configured to output the diagnosed risk as the diagnosis result.

When the diagnostic part represented in a color has a preset representation shape, the diagnostic part search unit may be configured to output a stop command for an ultrasound probe, and the diagnostic part represented in a color may be any one of a carotid artery part, a thyroid part, a breast part, a femoral vein part, and a medium vein part.

The diagnostic part search unit may include
a second artificial neural network pretrained to select only an ultrasound image of any one of a carotid artery, a thyroid, a breast, a femoral vein, and a medium vein from the input images and extract any one part of a carotid artery part, a thyroid part, a breast part, a femoral vein part, and a medium vein part from the selected ultrasound image to mark any one part with a virtual line, and the diagnostic part search unit may correct the virtual line using a brightness of a pixel and may be configured to represent and output any one part of the carotid artery part, the thyroid part, the breast part, the femoral vein part, and the medium vein part in a color differentiated from that of tissue.

When the diagnosis result is abnormal, the automatic diagnosis unit may diagnose a risk from an image of any one part of the carotid artery part, the thyroid part, the breast part, the femoral vein part, and the medium vein part using a pretrained third artificial neural network and may be configured to output the diagnosed risk as the diagnosis result. In such a system configuration, the diagnostic part search unit may include a second artificial neural network pretrained to select only an ultrasound image of any one of a carotid artery, a thyroid, a breast, a femoral vein, and a medium vein from the input images and extract any one part of a carotid artery part, a thyroid part, a breast part, a femoral vein part, and a medium vein part as the diagnostic part.

### Advantageous Effects of Invention

According to the above-described embodiments, since a carotid artery part, a thyroid part, a femoral vein part, a medium vein part, or a breast part is extracted from a carotid ultrasound image, a thyroid ultrasound image, a femoral vein ultrasound image, a medium vein ultrasound image, or a breast ultrasound image and is represented in a color, the present invention provides an advantage in that an operator of a diagnostic device can easily recognize the carotid artery part, thyroid part, femoral vein part, medium vein part, or breast part which is a diagnostic part.

Furthermore, the present invention provides convenience of guiding movement for an operation of a probe such that an ultrasound image of a diagnostic part necessary for automatic diagnosis can be acquired at an optimal position.

In addition, since only an ultrasound image of a carotid artery, a thyroid, a femoral vein, a medium vein, or a breast can be selected from input images through a pretrained artificial neural network to perform an automatic diagnosis, the present invention has an advantage in that inappropriate images, which cannot be diagnosed, can be filtered to increase the reliability of a system.

Through one or more artificial neural networks, whether a carotid artery, a thyroid, a femoral vein, a medium vein, or a breast is abnormal can be automatically diagnosed, and a risk and a lesion area of a diagnostic part can be marked together, thereby providing an effect of not only being dependent on a reading ability of each examiner (reader) but also accurately detecting even a symptom appearing subtly in an ultrasound image.

Furthermore, the present invention can be implemented as an independent ultrasound diagnostic device as well as a remote medical treatment server, thereby providing a remote medical service. In addition, since thrombi with high separability, which cannot be visually identified by an examiner, can be detected and represented in advance, examinees with the floating possibility of thrombi and examinees with carotid artery stenosis can take necessary measures in advance, and thus, a stroke risk can be prevented.

### BRIEF DESCRIPTION OF DRAWINGS

FIG. 1 is an exemplary block diagram of a medical diagnostic device including a carotid diagnostic system as an ultrasound diagnostic system according to one embodiment of the present invention.

FIG. 2 is an exemplary block diagram of a partial configuration of a carotid ultrasound diagnostic system according to another embodiment of the present invention in FIG. 1.

FIG. 3 is a flowchart for describing a diagnosis process of a carotid ultrasound diagnostic system according to one embodiment of the present invention.

FIGS. 4 to 10 are images for additionally describing the operation of the carotid ultrasound diagnostic system according to one embodiment of the present invention.

### DETAILED DESCRIPTION OF EMBODIMENT

In the following detailed description of the present invention, reference is made to the accompanying drawings that show, by way of illustration, specific embodiments in which the present invention may be practiced to clarify the objects, technical solutions, and advantages of the present invention. These embodiments are described in sufficient detail to enable those skilled in the art to practice the present invention.

It is to be understood by those skilled in the art that, throughout the detailed description and claims of the present invention, the term “learning” refers to deep learning performed according to a procedure and is not intended to refer to mental action such as human educational activities. In addition, it should be understood that, throughout the description and claims of the present invention, the word “comprise” and its variations are not intended to exclude other technical features, additions, components, or steps. Other objects, advantages, and features of the present invention will become apparent to those skilled in the art from the present specification, and in part from the practice of the present invention. The following examples and drawings are provided by way of illustration and are not intended to limit the present invention. Moreover, the present invention encompasses all possible combinations of embodiments shown herein. It should be understood that various embodiments of the present invention are different but need not be mutually exclusive. For example, certain features, structures, and characteristics described herein may be embodied in other embodiments without departing from the spirit and scope of the present invention in connection with one embodiment. It is also to be understood that the position or arrangement of the individual components within each disclosed embodiment may be varied without departing from the spirit and scope of the present invention. The following detailed description is, therefore, not to be taken in a limiting sense, and the scope of the present invention is to be limited only by the appended claims, along with the full scope of equivalents to those for which those claims are entitled, where properly explained. In the drawings, like reference numerals refer to the same or similar functions throughout the several views.

Unless otherwise indicated herein or clearly contradicted by context, an item referred to as singular is intended to encompass a plurality of items unless the context otherwise requires. Further, in describing the present invention, the detailed description of known related components or functions will be omitted when it may make the subject matters of the present invention unclear.

For reference, an artificial neural network to be described below may be, as an example, a convolutional neural network (CNN) model in which artificial neural networks are stacked in a multi-layer. The CNN model may be expressed as a deep neural network in the sense that the network has a deep structure. The deep neural network is trained in a method of learning a large volume of data to automatically learn each image and to minimize errors of an objective function. Since such a CNN model is already known, a detailed description thereof will be omitted.

In addition, hereinafter, embodiments of the present invention will be described by exemplifying a carotid ultrasound diagnostic system as an ultrasound diagnostic system according to an embodiment of the present invention.

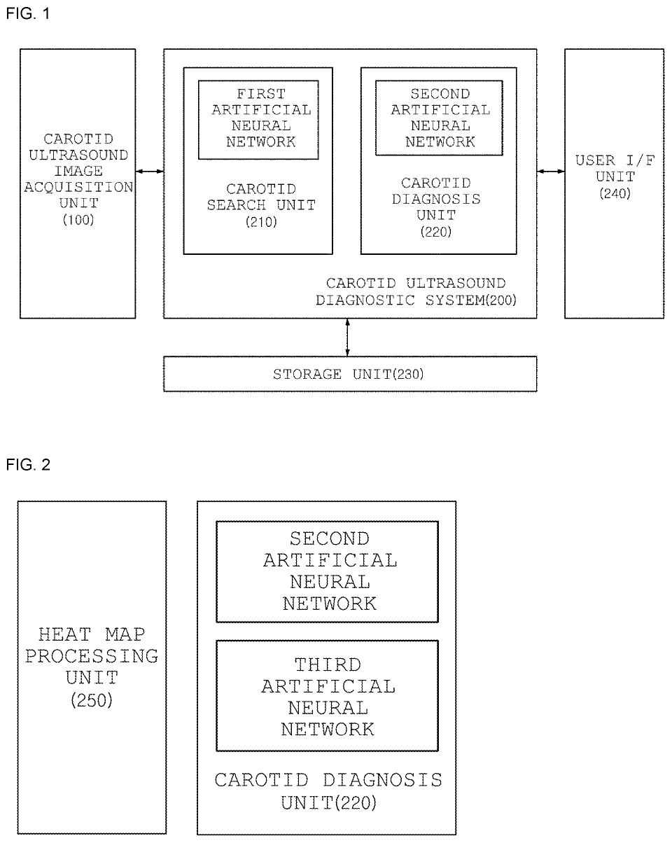

FIG. 1 is a block diagram of a medical diagnostic device including a carotid diagnostic system as an ultrasound diagnostic system according to one embodiment of the present invention, and FIG. 2 is a block diagram of a carotid diagnosis unit 220 which is a partial configuration of a carotid ultrasound diagnostic system according to another embodiment of the present invention in FIG. 1.

Among the terms used below, on the assumption of the carotid ultrasound diagnostic system, a carotid search unit 210 and the carotid diagnosis unit 220 are terms assigned to describe the embodiments of the present invention. For example, on the assumption of an ultrasound diagnostic system for diagnosing a femoral vein using ultrasound, the carotid search unit 210 may be referred to as a femoral vein search unit, and the carotid diagnosis unit 220 may be referred to as a femoral vein diagnosis unit. Accordingly, the carotid search unit, the femoral vein search unit, a medium vein search unit, a thyroid search unit, and a breast search unit are terms assigned according to diagnostic parts and thus will be referred to as diagnostic part search units in the claims of the present specification. In addition, the carotid diagnosis unit 220, the femoral vein diagnosis unit, the medium vein diagnosis unit, the thyroid diagnosis unit, and the breast diagnosis unit are configured to automatically diagnose whether diagnostic parts are abnormal, and thus will be referred to as automatic diagnosis units in the claims of the present specification.

Referring to FIG. 1, first, although FIG. 1 illustrates that a carotid ultrasound diagnostic system 200 according to the embodiment of the present invention constitutes a portion of a medical diagnostic device, for example, a portion of an ultrasound medical diagnostic device, the carotid ultrasound diagnostic system 200 may be constructed in a computer system that can perform a diagnosis by reading a carotid ultrasound image input, received, or read from a memory and may also be constructed in a remote diagnostic server that can be connected to a plurality of medical institution computer systems through a communication network for a remote diagnosis, thereby diagnosing whether a carotid artery is abnormal.

Referring to FIG. 1, a carotid ultrasound image acquisition unit 100 is configured to acquire an ultrasound image of a carotid artery to be diagnosed. When the carotid ultrasound diagnostic system 200 is a portion of a medical diagnostic device, the carotid ultrasound image acquisition unit 100 may be implemented to include an ultrasound probe which transmits an ultrasound signal to a diagnostic part including a carotid artery and receives an ultrasound echo signal reflected from the diagnostic part and an ultrasound image generation unit which processes the ultrasound echo signal provided from the ultrasound probe and converts the ultrasound echo signal into an ultrasound image of the carotid artery.

When the carotid ultrasound diagnostic system 200 is a diagnosis computer system used by a medical specialist or a computer system of a medical institution, the carotid ultrasound image acquisition unit 100 may be an interface unit capable of data-interfacing with peripheral ultrasound devices including the ultrasound probe and the ultrasound image generation unit and may be an interface unit capable of transmitting and receiving data to and from a portable storage device.

When the carotid ultrasound diagnostic system 200 is constructed in a remote diagnostic server, the carotid ultrasound image acquisition unit 100 may be a receiver for receiving a carotid ultrasound image from a computer system of a remote medical institution through a communication network.

Referring to FIG. 1 again, the carotid ultrasound diagnostic system 200 includes
the carotid search unit 210 (may be referred to as a diagnostic part search unit) which finds a carotid artery part (corresponding to a diagnostic part) from an input image and is configured to represent and output at least the carotid artery part (diagnostic part) in a color differentiated from that of tissue, and
the carotid diagnosis unit 220 (may be referred to as an automatic diagnosis unit) which diagnoses whether a carotid artery is abnormal from an image of the carotid artery part found by the carotid search unit 210 based on a second artificial neural network and is configured to output a diagnosis result.

The carotid search unit 210 may include a first artificial neural network pretrained to select only a carotid ultrasound image from input images and to extract a carotid artery part from the selected carotid ultrasound image.

In order to select only a carotid ultrasound image from input images, the first artificial neural network may use a 2-class classification artificial intelligence algorithm to learn a carotid ultrasound image at one side in advance and learn general object images (desk, traffic light, and sofa images), which are not the carotid ultrasound image, as well as a carotid ultrasound image, a thyroid ultrasound image, and an abdominal ultrasound image, which are inappropriate for a diagnosis, at the other side in advance, thereby selecting only the carotid ultrasound image effective for a diagnosis from the input images.

According to an implementation method, the first artificial neural network may also be configured to primarily select only an ultrasound image from input images, select only a carotid ultrasound image from the selected ultrasound image, and then filter a carotid ultrasound image that is inappropriate for a diagnosis.

Furthermore, the first artificial neural network included in the carotid search unit 210 may be an artificial neural network trained to select and display both a carotid ultrasound image in a longitudinal direction (B-Type) and a carotid ultrasound image in a lateral direction (A-Type). The lateral direction refers to a direction in which, when a blood vessel ascending from a neck to a brain of a person is cut in a cross-sectional direction, a cross section of the blood vessel is visible, and the longitudinal direction refers to a length direction of the blood vessel.

As another modified embodiment, the carotid search unit 210 includes
the first artificial neural network pretrained to select only a carotid ultrasound image from input images, extract a carotid artery part from the selected carotid image, and mark the extracted carotid artery part with a virtual line. In this case, the carotid search unit 210 may correct the virtual line using a brightness of a pixel, thereby more smoothly finishing roughness formed between an edge of a carotid artery and tissue.

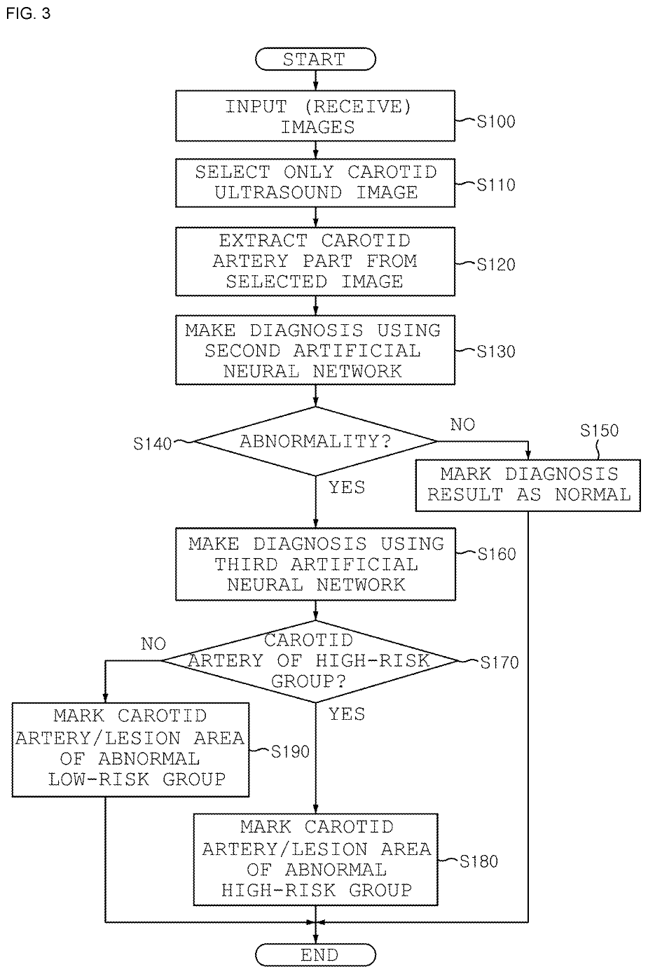

Correcting a virtual line using a brightness of a pixel and representing a carotid artery part in a color differentiated from that of tissue will be described in more detail with reference to FIG. 3.

As still another modified embodiment, when a carotid artery part represented in a color has a preset representation shape, the carotid search unit 210 may be configured to output a stop command for the ultrasound probe, thereby guiding a user of the ultrasound probe to acquire a carotid ultrasound image at an optimal position. This will also be described in more detail with reference to FIG. 3.

The above-described first artificial neural network may be trained by setting an ultrasound carotid image or a carotid artery part marked by a medical specialist as learning data.

Meanwhile, the carotid diagnosis unit 220 including the second artificial neural network trains the second artificial neural network by setting one or more images of a carotid artery part marked as normal or abnormal by a medical specialist as learning data.

The carotid diagnosis unit 220 may mark a lesion area in an image of a carotid artery part and may be configured to output the marked lesion area as a diagnosis result.

As a modified embodiment, as shown in FIG. 2, the carotid diagnosis unit 220 may further include a third artificial neural network in addition to the second artificial neural network. When a diagnosis result by the second artificial neural network is abnormal, the carotid diagnosis unit 220 may diagnose a carotid artery risk with respect to an image of a carotid artery part using the pretrained third artificial neural network and may be configured to output the diagnosed risk to a display unit constituting a user interface (I/F) unit as a diagnosis result.

The “carotid artery risk” refers to a risk marked by grading a risk according to stages such as grading into an “abnormal high-risk group” and an “abnormal low-risk group.” Although, in the following description, a risk is graded into two stages, this is merely an example, and the stages may be subdivided into two or more stages.

The carotid diagnosis unit 220 including the second and third artificial neural networks trains the third artificial neural network by setting a lesion area set in one or more images of a carotid artery part set by a medical specialist and a carotid artery risk for the lesion area as learning data. For example, a medical specialist reads an abnormal carotid artery part to set a lesion area, in which plaque is excessively positioned, with a box, set a lesion area, in which a thrombus is excessively distributed in a blood vessel, with a box, set a lesion area, in which the separability of a thrombus is high, with a box, and set a lesion area, in which carotid artery stenosis is visible, with a box, thereby setting a carotid artery risk for each set lesion area, for example, setting a carotid artery of an abnormal high-risk group and a carotid artery of an abnormal low-risk group together. Then, when the medical specialist gives a command for learning a lesion area and a carotid artery part to which a carotid artery risk is set, the carotid diagnosis unit 220 trains the third artificial neural network by setting the lesion area and the carotid artery part, to which the carotid artery risk is set, as learning data.

When the third artificial neural network is trained in such a manner, it is possible to automatically diagnose not only a lesion area but also carotid artery risk information of a carotid artery part that is primarily diagnosed as an abnormal carotid artery in a subsequent diagnosis mode.

Meanwhile, the carotid diagnosis unit 220 shown in FIG. 2 may mark and output a carotid artery risk as well as a lesion area (for example, a position of plaque) together within an image of a carotid artery part as a diagnosis result.

Furthermore, the carotid diagnosis unit 220 shown in FIG. 2 may expand a carotid artery part before diagnosing a carotid artery part image using the third artificial neural network.

In addition, the carotid ultrasound diagnostic system 200 shown in FIGS. 1 and 2 may further include a heat map processing unit 250 which increases visibility of a carotid artery part image extracted from the carotid search unit 210, processes the carotid artery part image with a heat map in order to increase diagnostic performance, and transfers the heat map image to the carotid diagnosis unit 220. The heat map processing unit 250 may be implemented as being included in the carotid diagnosis unit 220.

For reference, in the carotid ultrasound diagnostic system 200 according to the embodiment of the present invention, the first artificial neural network, the second artificial neural network, and the third artificial neural network can be combined to construct various types of carotid ultrasound diagnostic system 200. In addition, the heat map processing unit 250 can be further included in the various types of constructed system to increase visibility and diagnostic performance. For reference, according to experimental values, when a heat map image was used, sensitivity appeared to be generally improved as compared with a grayscale image.

A storage unit 230 in FIG. 1 includes a database (DB) which stores control program data necessary for the carotid ultrasound diagnostic system 200 to control the overall operation of a medical device as well as learning data set by a medical specialist and settings related to each learning data or pieces of marking information.

The user I/F unit 240 includes an operation unit through which a medical specialist sets an environment, an operation mode, an ROI, and the like of the carotid ultrasound diagnostic system 200 and a display unit which displays a variety of display data according to a system operation, diagnosis results, and carotid ultrasound images acquired by the ultrasound probe and the like.

Hereinafter, the operation of the carotid ultrasound diagnostic system 200, which may have the above-described configuration and various combinations of artificial neural networks, will be described in more detail with reference to the accompanying drawings. Hereinafter, diagnosing whether a carotid artery is abnormal by analyzing a longitudinal carotid ultrasound image will be described.

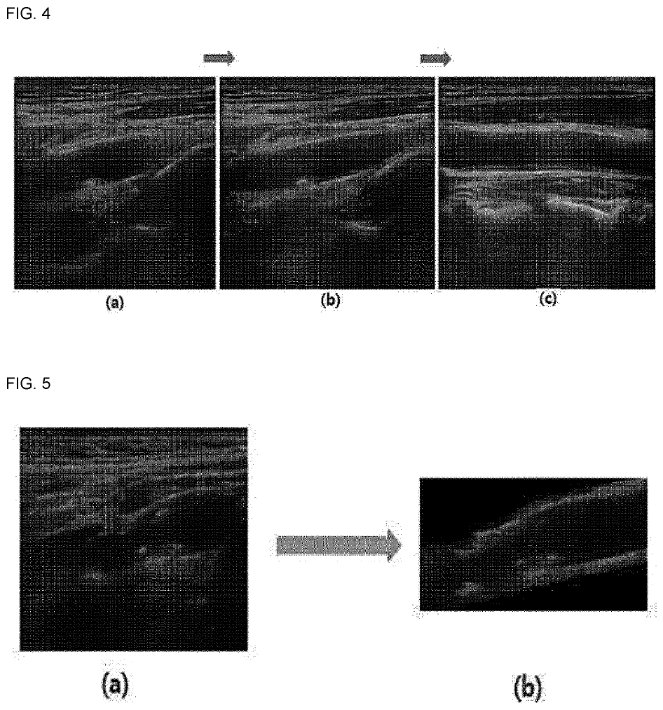

FIG. 3 is a flowchart for describing a diagnosis process of a carotid ultrasound diagnostic system according to an embodiment of the present invention, and FIGS. 4 to 10 are images for additionally describing the operation of the carotid ultrasound diagnostic system according to the embodiment of the present invention.

Referring to FIG. 3, first, in a diagnosis mode, an image may be input through a carotid ultrasound image acquisition unit 100 (operation S100). The input image is input to a carotid search unit 210.

The carotid search unit 210 selects only a carotid ultrasound image from the input images using a pretrained first artificial neural network (operation S110). As described above, in a learning mode, the first artificial neural network may use a 2-class classification artificial intelligence algorithm to learn a carotid ultrasound image and general object images (desk, traffic light, and sofa images) or other ultrasound images in advance, thereby selecting only the carotid ultrasound image effective for a diagnosis from the input images. FIG. 4 shows selected carotid ultrasound images.

Thereafter, the carotid search unit 210 extracts a carotid artery part from the selected carotid ultrasound image (operation S120). For reference, in the learning mode, a blood vessel part marked by a medical specialist is learned from a readable carotid ultrasound image. In an entire carotid ultrasound image in grayscale, since white represents a blood vessel, and black represents tissue, when a medical specialist marks only the blood vessel in a curved shape, such a pattern may be learned, thereby extracting a carotid artery part to draw a virtual line at a boundary between the extracted carotid artery part and the tissue as shown in FIG. 5B.

Then, the carotid search unit 210 represents the carotid artery part divided by the virtual line in a color differentiated from that of the tissue. A reason and an example of representing the extracted carotid artery part in a different color so as to be differentiated from the tissue as described above will be described below.

However, since the virtual line is generated by learning a pattern marked in a curved shape by a medical specialist, a roughness of the line may be high and a shape of the line may be unnatural.

Thus, as a modified embodiment, the carotid search unit 210 may adopt a method in which a brightness of pixels positioned within a predetermined area based on the virtual line is used to smoothly correct the virtual line and to represent the carotid artery part in a color differentiated from that of the tissue.

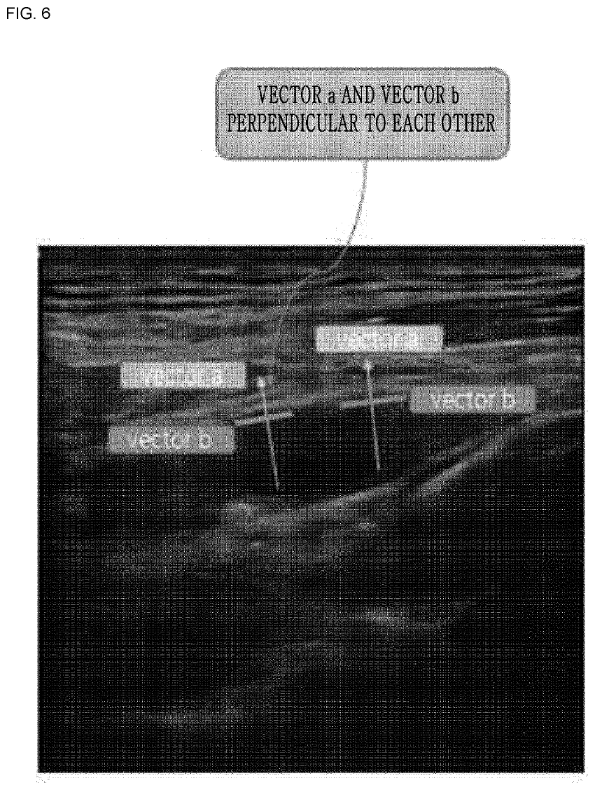

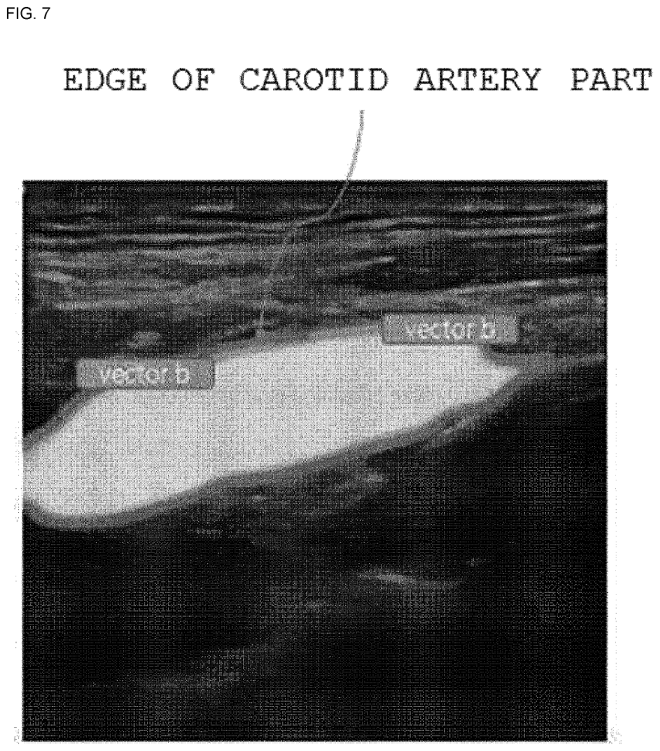

More specifically, an edge between a blood vessel and tissue may be a point at which brightness is changed the most (a direction in which a value is changed the most in a grayscale image may be defined as the edge), which corresponds to a direction in which a differential value is mathematically changed the most at one point. Since an image is in a pixel unit, when a point at which brightness is changed the most in one pixel, that is, a direction in which a differential value is the greatest at the point is defined as a vector a as shown in FIG. 6, a vector b perpendicular to the vector a may be defined. In addition, as shown in FIG. 7, all of the vectors b are connected to obtain a carotid artery part having an edge in which a virtual line is smoothly corrected.

Accordingly, the carotid search unit 210 may represent a carotid artery part having an edge, in which a virtual line is smoothly corrected, in a color differentiated from that of tissue as shown in FIG. 7.

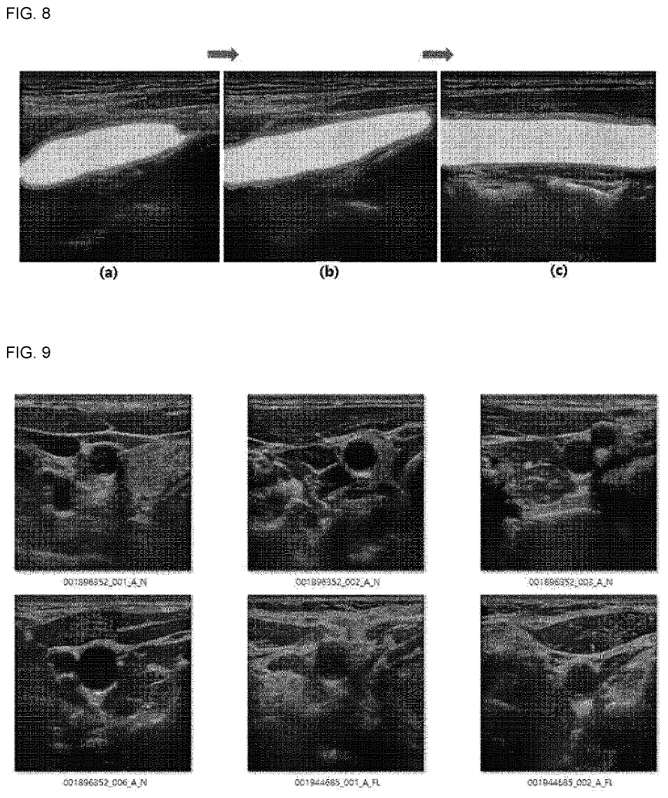

FIG. 8 shows carotid artery parts that each correspond to one of the carotid ultrasound images shown in FIGS. 4A, 4b, and 4C and are represented in a color. Even when an operator of an ultrasound probe does not have specialized knowledge like a medical specialist, as shown in FIGS. 8A, 8B, and 8C, while viewing a display screen, the operator can recognize the carotid artery part and can move the ultrasound probe to a position (see FIG. 8C) at which an optimal carotid ultrasound image can be obtained.

For reference, the difficulty that the operator of the ultrasound probe faces during a carotid ultrasound test is that the operator should recognize a diagnostic part and should stop the ultrasound probe at a specific position (represented as a frame image) in order to acquire an optimal readable image.

In order to solve such a problem, since the carotid search unit 210 according to the embodiment of the present invention represents a carotid artery part in a color as shown in FIG. 8, the operator of the probe can easily recognize the carotid artery part that is a diagnostic part.

Furthermore, when a carotid artery part represented in a color has a preset representation shape as shown in FIG. 8C (when a rectangular carotid artery is well represented to be laterally elongated), the carotid search unit 210 may be configured to output a stop command for the ultrasound probe. Thus, such a problem can be solved in such a manner that the operator of the probe operator moves the ultrasound probe until an image as shown in FIG. 8C is acquired, and when the stop command is output, the operator may stop the operation of the probe. For reference, for a lateral image of carotid ultrasound, it is common to stop the ultrasound probe when a blood vessel is represented in a circular shape.

Accordingly, the present invention provides convenience capable of guiding the operator of the probe to acquire a carotid ultrasound image at an optimal position. When the ultrasound diagnostic system according to the embodiment of the present invention is a femoral vein or medium vein ultrasound diagnostic system, the ultrasound diagnostic system provides convenience capable of guiding the operator of the probe to acquire a femoral vein or medium vein ultrasound image at an optimal position through the above-described method.

As described above, the ultrasound probe is stopped at an optimal position, and an image of a carotid artery part extracted from a carotid ultrasound image acquired from the ultrasound probe is transferred to the carotid diagnosis unit 220. Noise may be removed from the image of the carotid artery part through a denoising process operation.

The carotid diagnosis unit 220 may simply diagnose based on machine learning whether a carotid artery is abnormal with respect to the transferred image of the carotid artery part and may be configured to output a diagnosis result.

In some cases, the carotid diagnosis unit 220 may diagnose whether a carotid artery of the carotid artery part is abnormal using a pretrained second artificial neural network (operation S130). When a diagnosis result is normal (operation S140), the carotid diagnosis unit 220 marks the diagnosis result as normal on the user I/F unit 240 (operation S150) and ends a series of diagnosis processes.

When the diagnosis result is abnormal and when the carotid diagnosis unit 220 does not include a third artificial neural network, the carotid diagnosis unit 220 may simply mark the diagnosis as abnormal. On the other hand, when the carotid diagnosis unit 220 includes the third artificial neural network, the carotid diagnosis unit 220 may expand the carotid artery part before diagnosing a carotid artery part image using the third artificial neural network. Such expansion of the carotid artery part is one possible option.

The carotid diagnosis unit 220 diagnoses a carotid artery risk for the extracted carotid artery part using the pretrained third artificial neural network (operation S160). As a diagnosis result, when the carotid artery risk is a risk for a carotid artery of a high-risk group (operation S170), the procedure proceeds to operation S180, and a carotid artery and a lesion area of an abnormal high-risk group (FH) are marked (with a bounding box). When the carotid artery risk is a risk for a carotid artery of a low-risk group, the procedure proceeds to operation S190, a carotid artery and a lesion area of an abnormal low-risk group (FL) are marked (with a bounding box), and then, a series of diagnostic procedures are ended.

As described above, in the carotid ultrasound diagnostic system 200 according to the embodiment of the present invention, a carotid artery part is found from a carotid ultrasound image input or transmitted through the carotid ultrasound image acquisition unit 100 or read from a memory through the first artificial neural network, and at least the carotid artery part is output by being represented in a color differentiated from that of tissue, thereby guiding an operator of a probe to acquire a carotid ultrasound image at an optimal position.

Furthermore, through one or more artificial neural networks, whether a carotid artery part is abnormal is automatically diagnosed, and a risk and a lesion area of a carotid artery are marked together, and thus the carotid ultrasound diagnostic system 200 according to the embodiment of the present invention is a useful invention that can provide an effect of not only being dependent on a reading ability of each examiner (reader) but also accurately detecting even a symptom appearing subtly in an ultrasound image.

Furthermore, the present invention can be implemented as an independent ultrasound diagnostic device as well as a remote medical treatment server, thereby providing a remote medical service. In addition, since thrombi with high separability, which cannot be visually identified by an examiner, can be detected and represented in advance, examinees with the floating possibility of thrombi and examinees with carotid artery stenosis can take necessary measures in advance, and thus, a stroke risk can be prevented.

Although in the above-described embodiment of the present invention, it has been described that a rectangular carotid ultrasound image is learned to automatically diagnose whether a carotid artery is abnormal, as shown in FIG. 9, a lateral carotid ultrasound image may be learned to represent a carotid artery part in a color differentiated from that of tissue and also automatically diagnose whether a carotid artery is abnormal, and carotid ultrasound images in both a longitudinal direction and a lateral direction may also be learned to automatically diagnose whether a carotid artery is abnormal. In some cases, when results of automatically diagnosing whether a carotid artery is abnormal with respect to carotid ultrasound images in both a longitudinal direction and a lateral direction do not match each other, through a newly trained artificial neural network, whether the carotid artery is abnormal may be finally diagnosed with respect to any one image which is diagnosed to be normal or abnormal.

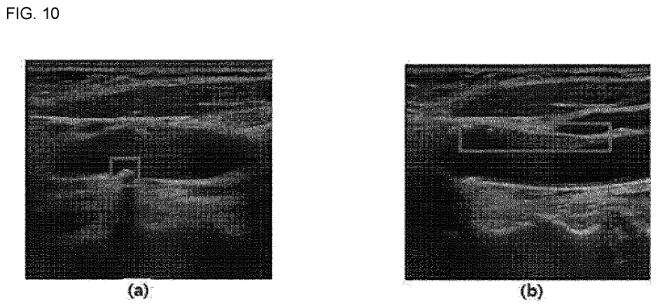

In addition, the carotid diagnosis unit 220 of the present invention may vary and represent a size of a bounding box around a lesion area marked by a medical specialist or a lesion (plaque) area detected as a diagnosis result as shown in FIG. 10.

It can be clearly understood by those skilled in the art that, based on the description of the above embodiments, the present invention may be accomplished by a combination of software and hardware or by hardware alone. The objects of the technical solutions of the present invention or portions contributed to the related art may be embodied in the form of program instructions that can be executed through various computer components and recorded on a computer-readable recording medium. The computer-readable recording medium may include program instructions, data files, data structures, and the like, alone or in combination. The program instructions recorded on the computer-readable recording medium may be those specially designed and constructed for the present invention or may be those known to those skilled in the art of computer software.

Examples of the program instructions include a machine language code such as that generated by a compiler, as well as a high-level language code that can be executed by a computer using an interpreter or the like. A hardware device may be configured to operate as one or more software modules for performing the process according to the present invention, and vice versa. The hardware device may include a processor, such as a central processing unit (CPU) or a graphics processing Unit (GPU), connected to a memory shown in FIG. 1, such as read only memory (ROM) or random access memory (RAM) for storing program instructions, and configured to execute the instructions stored in the memory and may include a communication unit that can transmit and receive signals to and from an external device. In addition, the hardware device may include a keyboard, a mouse, and other external input devices for receiving instructions prepared by developers.

While the present invention has been described with reference to the limited embodiments and the accompanying drawings along with specific items such as particular components and the like, the embodiments set forth herein are provided only to aid in overall understanding of the present invention and are not intended to limit the scope of the present invention. In addition, from the foregoing description, it will become apparent to those of ordinary skill in the art to which the present invention pertains that various changes and modification can be made.

For example, although in the embodiments of the present invention, it has been described that an automatic diagnosis is performed on an ultrasound image of a carotid artery, abnormal symptoms of thyroid, femoral vein, medium vein, and breast parts can be automatically diagnosed with respect to ultrasound images of a thyroid, a femoral vein, a medium vein, and a breast without any modification, of course, an abnormal symptom can be automatically diagnosed for each diagnostic part, but it is possible to construct a system in which abnormal symptoms of thyroid, femoral vein, medium vein, and breast parts may be learned in advance in one system to automatically diagnose an abnormal symptom of a diagnostic part even when an ultrasound image of any one of a thyroid, a femoral vein, a medium vein, and a breast is input. Accordingly, the spirit of the present invention should not be limited and defined by the described embodiments, and it may be said that the claims to be described below and all of things equally or equivalently modified from respect to the claims belong to the category of the spirit of the present invention.

What is claimed is:

1. An ultrasound diagnostic system comprising:
a diagnostic part search unit which finds a diagnostic part from input images and is configured to represent and output at least the diagnostic part in a color differentiated from that of tissue; and
an automatic diagnosis unit which diagnoses whether the diagnostic part is abnormal with respect to an image of the diagnostic part found by the diagnostic part search unit based on a first artificial neural network and is configured to output a diagnosis result.

2. The ultrasound diagnostic system of claim 1, wherein the diagnostic part search unit includes a second artificial neural network pretrained to select only an ultrasound image of any one of a carotid artery, a thyroid, a breast, a femoral vein, and a medium vein from the input images and extract any one part of a carotid artery part, a thyroid part, a breast part, a femoral vein part, and a medium vein part from the selected ultrasound image.

3. The ultrasound diagnostic system of claim 2, wherein the second artificial neural network is trained to select and display carotid ultrasound images in both a longitudinal direction and a lateral direction.

4. The ultrasound diagnostic system of claim 2, wherein, when the diagnosis result is abnormal, the automatic diagnosis unit diagnoses a risk from an image of any one part of the carotid artery part, the thyroid part, the breast part, the femoral vein part, and the medium vein part using a pretrained third artificial neural network and is configured to output the diagnosed risk as the diagnosis result.

5. The ultrasound diagnostic system of claim 2, wherein the automatic diagnosis unit marks a lesion area in an image of any one part of the carotid artery part, the thyroid part, the breast part, the femoral vein part, and the medium vein part and is configured to output the marked lesion area together with the diagnosis result.

6. The ultrasound diagnostic system of claim 1, wherein, when the diagnostic part represented in a color has a preset representation shape, the diagnostic part search unit is configured to output a stop command for an ultrasound probe, and
the diagnostic part represented in a color is any one of a carotid artery part, a thyroid part, a breast part, a femoral vein part, and a medium vein part.

7. The ultrasound diagnostic system of claim 1, wherein the diagnostic part search unit includes a second artificial neural network pretrained to select only an ultrasound image of any one of a carotid artery, a thyroid, a breast, a femoral vein, and a medium vein from the input images and extract any one part of a carotid artery part, a thyroid part, a breast part, a femoral vein part, and a medium vein part from the selected ultrasound image to mark any one part with a virtual line, and
the diagnostic part search unit corrects the virtual line using a brightness of a pixel and is configured to represent and output any one part of the carotid artery part, the thyroid part, the breast part, the femoral vein part, and the medium vein part in a color differentiated from that of tissue.

8. The ultrasound diagnostic system of claim 7, wherein, when the diagnosis result is abnormal, the automatic diagnosis unit diagnoses a risk from an image of any one part of the carotid artery part, the thyroid part, the breast part, the femoral vein part, and the medium vein part using a pretrained third artificial neural network and is configured to output the diagnosed risk as the diagnosis result.

9. The ultrasound diagnostic system of claim 1, wherein, when the diagnosis result is abnormal, the automatic diagnosis unit diagnoses a risk from an image of the diagnostic part found by the diagnostic part search unit using a pretrained third artificial neural network and is configured to output the diagnosed risk as the diagnosis result.

10. The ultrasound diagnostic system of claim 9, wherein the diagnostic part search unit includes a second artificial neural network pretrained to select only an ultrasound image of any one of a carotid artery, a thyroid, a breast, a femoral vein, and a medium vein from the input images and extract any one part of a carotid artery part, a thyroid part, a breast part, a femoral vein part, and a medium vein part as the diagnostic part.

11. The ultrasound diagnostic system of claim 10, wherein the automatic diagnosis unit marks a lesion area in an image of any one part of the carotid artery part, the thyroid part, the breast part, the femoral vein part, and the medium vein part and is configured to output the marked lesion area together with the diagnosis result.
